(19) United States
(12) Patent Application Publication
(10) Pub. No.: US 2009/0299053 A1
(43) Pub. Date: Dec. 3, 2009

(54) Product Resulting From the Grafting of Fatty Chains to Ulvans and Use of Said Product as a Surfactant

(75) Inventors: Michèle Ranson, Paris (FR); Gilles Guitierrez, Rabat (MT); Dominique Brault, Lezardieux (FR); Herve Le Deit, Lannion (FR); Xavier Pages-Xatart-Pares, Cestas (FR); Carine Alfos, Pessac (FR)

Correspondence Address: HAMRE, SCHUMANN, MUELLER & LARSON, P.C., P.O. BOX 2902, MINNEAPOLIS, MN, 55402-0902, US

(73) Assignees: JAVENECH, Javene (FR); ENVIRONMENTAL PROTECTION ENGINEERING S.A., Piraeus (GR); ICP INSTITUTE FOR CELLULAR PHARMACOLOGY, Mosta (MY)

(21) Appl. No.: 12/083,702

(22) PCT Filed: Oct. 18, 2006
(86) PCT No.: PCT/FR2006/051055

§ 371 Date: Aug. 19, 2009

(30) Foreign Application Priority Data
Oct. 19, 2005 (FR) 0510643

(51) Int. Cl. C07H 7/00; C07H 1/00

(52) U.S. Cl. 536/123.1

(57) ABSTRACT

The invention relates to a product resulting from the grafting, by esterification or transesterification, onto at least a part of the hydroxyl functions of an ulvan-type polysaccharide in the form of an acid or in the form of a mono- or divalent salt, in particular a sodium salt, of fatty chains or of mixtures of fatty chains containing 8 to 28 carbon atoms, said fatty chains being saturated or unsaturated, and linear or branched.

It also relates to the method for preparing this product.

It also relates to uses of this product, in particular as surfactant.

The present invention relates to novel ulvan derivatives, to the methods for preparing them, and to the uses thereof, in particular as surfactants.

It is known that marine algae are an important source of polysaccharides with gelling and thickening properties, widely used for dietary and nondietary purposes.

Ulvans are extracted from ulvas or enteromorphs. These green-colored algae are part of the phylum Chlorophyta and of the order Ulvales, characterized by a tubular thallus (enteromorph) or a thallus made up of a double cell layer (ulva). Several species of ulvas exist, in particular Ulva lactuca, Ulva rigida, Ulva armoricana and Ulva rotundata, and also several species of enteromorphs, such as, in particular, Enteromorpha compressa, Enteromorpha intestinalis and Enteromorpha ramulosa.

Various methods for extracting ulvans from algae have been described in the literature.

Mention will most particularly be made of the method described by M. Lahaye, B. Ray, S. Baumberger, B. Quemener and M. A. V. Axelos in Hydrobiologia 326/327:473-480, 1996.

According to this method, a fresh or dried alga is ground and then solubilized in water and brought to reflux for one hour. The suspension is subsequently centrifuged and then re-extracted a second time according to the same protocol. The two supernatants are subsequently combined and the ulvans are then precipitated with ethanol. After filtration, the ulvans are subsequently oven-dried. The yields are of the order of 10% relative to the solids content of the starting alga.

Ulvans are water-soluble, sulfated, anionic polysaccharides. In the cell, they are located in the cell wall. The literature (Lahaye M., Jegou D. Buleon A. E. Carbohydr Res 1994; 262:115) indicates that ulvans are of the sulfated xyloramnoglucoronan or sulfated glucoronorhamnoxyloglycan family.

The sugars which go to make up the composition of ulvans are rhamnose sulfated in the 3-position, galactose, glucose and xylose in terms of neutral sugars, and glucuronic acid and iduronic acid in terms of acidic sugars (Lahaye M., Alvarez-Cabal Cimadevilla E., Kuhlenkamp R., Quemener B. Lognoné V., Dion P.; Journal of Applied Phycology 11: 1-7, 1999).

The xylose may be partially sulfated.

The relative proportion of the sugars is variable depending on the site from which the ulvas were harvested, the species and also the harvesting time during the year. The following table gives sugar contents recorded in various ulva species:

| | | | | | | |
|---|---|---|---|---|---|---|
| | | | | | Glucuronic | Iduronic |
| Samples | Rhamnose | Galactose | Glucose | Xylose | acid | acid |

Ulva armoricana

| | | | | | | |
|---|---|---|---|---|---|---|
| 1993 Saint | 48.3 | 3.1 | 17.5 | 10.1 | 15.2 | 5.9 |
| Brieuc | | | | | | |
| 9/94 Saint | 51.6 | 1.2 | 8.4 | 9.1 | 22.8 | 7.0 |
| Brieuc | | | | | | |
| 10/94 Binic | 53.6 | 1.0 | 6.1 | 7.0 | 25.5 | 6.9 |
| 4/95 Saint | 47.8 | 1.3 | 13.0 | 8.2 | 25.9 | 3.8 |
| Brieuc | | | | | | |

Ulva ridiga

| | | | | | | |
|---|---|---|---|---|---|---|
| 9/94 | 50.9 | 1.3 | 6.6 | 7.9 | 28.9 | 4.4 |
| Pleubian | | | | | | |
| 10/94 | 52.2 | 0.9 | 5.3 | 6.5 | 30.4 | 4.7 |
| Roscoff | | | | | | |
| 5/95 Etang | 58.3 | 1.7 | 5.0 | 12.0 | 19.0 | 4.0 |
| de Pau | | | | | | |

Ulva rotundata

| | | | | | | |
|---|---|---|---|---|---|---|
| 9/94 | 49.6 | 1.5 | 5.4 | 23.8 | 17.8 | 2.0 |
| Pleubian | | | | | | |
| 10/96 le | 46.7 | 3.0 | 14.4 | 15.4 | 20.0 | 0.6 |
| Palmones | | | | | | |

The green algae of the ulva family, including the species produced by the green tides, contain ulvans which are water-soluble complex polysaccharides comprising both sulfated monosaccharides and uronic acids. The chemical structure of ulvans is based on the repetition of various disaccharide units. The disaccharide units are predominantly composed of sodium ulvanobiuronate 3-sulfate type A comprising rhamnose 3-sulfate linked to glucuronic acid via a 1→4-type linkage, and sodium ulvanobiuronate 3-sulfate type B comprising rhamnose 3-sulfate linked to iduronic acid via a 1→4-type linkage:

The two disaccharide units are represented below.

Ulvanobiuronic acid 3-sulfate A disaccharide unit

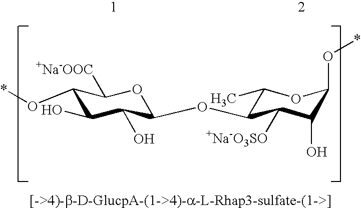

Ulvanobiuronic acid 3-sulfate B disaccharide unit

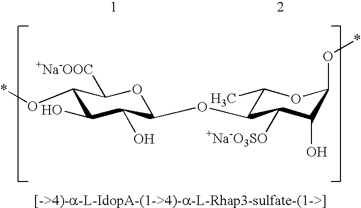

The other minority sugars (xylose, galactose and glucose) are inserted on the chain in an indeterminate manner and certainly in a variable manner according to the species, the locations and the periods of the year.

The polysaccharide may also bear side chains, the structure of which has not been clearly identified.

The literature mentions a large number of documents concerning the synthesis or the use of polyose-esters. Mention will in particular be made of U.S. Pat. No. 4,517,360 and U.S. Pat. No. 3,963,699, which describe polyose-esters derived from mono- and disaccharides or from polyols of sucrose, xylitol or sorbitol type, and also French patent 2 009 161, which describes polyol-esters derived from natural starches. International application WO 92/13006 also describes complex products based on polyose and fatty acids resulting from the reaction of at least one polyose with at least one fatty acid, in particular in halide or anhydride form.

However, none of the documents of the literature suggests using, as polyose, a polysaccharide of ulvan type and, even less, the advantage, as surfactant, provided by the product of esterification of such a polysaccharide and the additional advantage that would be provided by the development, in the form of products with a surfactant nature, of ulva-type algae rather considered essentially to date as nuisances, their proliferation resulting essentially from pollution.

In fact, the green tide phenomenon each year affects certain beaches, in particular of Brittany (Côtes d'Armor) and causes on said beaches a nuisance in terms of smell and appearance. This hinders the tourism activity (second largest economic sector of the department of Côtes d'Armor) and generates high gathering costs. From the 1980s onward, the gathering of green algae proved to be necessary in order to maintain the tourism activity of the sites and to overcome the nuisance generated by this phenomenon in terms of smell and appearance. Thus, in 2000, more than 75 000 m³ of green algae were the subject of collection on the Brittany coasts. The algae are then stored in uncontrolled dumps, or directly spread on agricultural land. The factors responsible for the green tides are today known. They lie in the drainage basins which support intensive agriculture generating nutritive salts, which end up in excess in the coastal waters.

Thus, an additional advantage of the invention is to develop a product normally considered to be a nuisance.

The invention proposes novel products which result directly from the grafting, by esterification or transesterification, of fatty chains onto ulvans.

The particularly advantageous properties of the products of the invention make it possible to envision them being used in many fields, as surfactants, in particular with a view to the preparation of emulsions. As will emerge from the description and the examples which follow, properties are particularly advantageous in various fields, in particular in the cosmetics field, due to the particularly soft and substantive feel of these products and also their emulsifying and hydrating properties, but also in completely different fields, such as the recovery or dispersion of hydrocarbons.

More specifically, according to one of its essential features, the invention relates to a product resulting from the grafting, by esterification or transesterification, onto at least a part of the hydroxyl functions of a polysaccharide of ulvan type in the form of an acid or in the form of a salt of a monovalent or divalent cation, in particular a sodium salt, of fatty chains or of mixtures of fatty chains containing 8 to 28 carbon atoms, said fatty chains being saturated or unsaturated, and linear or branched.

This product, hereinafter referred to as “polyose-ester” is derived from the reaction of polysaccharides (ulvans) extracted from algae of ulva or enteromorph type or from mixtures of these two types of algae, with compounds comprising fatty chains containing between 8 and 28 carbon atoms, it being possible for these chains to be saturated or unsaturated, and linear or branched.

For the preparation of the ulvan by extraction from the ulva, reference will be made to a method of extraction described in Carbohydrate Research 274 (1995), 251-261, or in Hydrobiologia 326/327: 473-480, 1996.

In general, the extracts used according to the invention are advantageously prepared by extraction of ulvas and/or enteromorphs in an aqueous medium, the extraction step being advantageously followed by an ultrafiltration step.

For the purpose of the invention, the expression “degree of grafting of fatty chains onto the ulvan backbone” is intended to mean the proportion by weight of fatty acid relative to the starting polyose.

Those skilled in the art will understand that, depending on the nature of the fatty chains and on the degree of grafting of these chains onto the polysaccharide backbone, properties that will vary from one product to the other will be obtained. It will thus be possible to vary in particular the rheological, solubilizing, gelling, thickening, emulsifying, coemulsifying and organoleptic properties, and also the solubility, lipophilicity, texture, color, surface tension, interfacial tension, critical micellar concentration in solution, saponification index, iodine index and acidity of the products of the invention.

By way of example, and in order to facilitate the understanding of the type of reaction involved, two chemical formulae of grafted disaccharide units are given below, each group R representing either a carboxylated fatty chain or a hydrogen and being independent of the others, provided that the disaccharide unit contains at least one fatty chain.

Grafted disaccharide unit of ulvanobiuronic acid 3-sulfate A

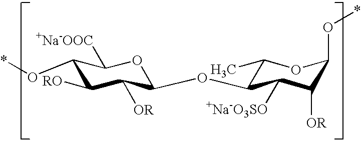

Grafted disaccharide unit of ulvanobiuronic acid 3-sulfate B

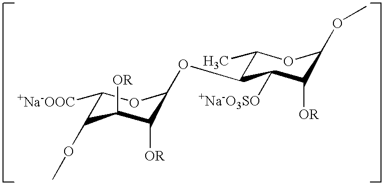

According to a second essential feature of the invention, it relates to a method for preparing the products and mixtures of products designated above.

In general, this method comprises an esterification or transesterification step using at least a part of the hydroxyl functions of a polysaccharide of ulvan type or of a salt thereof, obtained by extraction from an ulva-type alga.

Various variants of this method can be envisioned, in particular in order to take into account the specific nature of the backbone onto which it is desired to graft the fatty chains, and the degree of grafting that it is desired to carry out, which degree can vary in particular with the intended field of application.

Thus, the various methods developed by the inventors of the present invention are all based on methods described in the literature, taking into account both the specific nature of the polyose substrate and the intended application which may require a greater or lesser purity of the product and/or a greater or lesser degree of grafting of the fatty chains onto the polysaccharide backbone.

Thus; it will be possible to envision various variants of methods, the common point of which will be to carry out an esterification or a transesterification of fatty chains on an extract of ulva or of enteromorph in solid form, generally containing at least 85% by weight of ulvans, preferably at least 95%, it being possible for this extract to in particular be obtained according to the protocol described in Hydrobiologia 326/327: 473-480, 1996.

According to a first variant of the method of the invention, the grafting will be carried out by esterification, in particular by means of an acid chloride of an acid comprising a fatty chain containing from 8 to 28 carbon atoms.

Such a method has the advantage of being able to prepare highly grafted products in which the proportion by weight of fatty acid relative to the polyose is at least equal to 10%, preferably between 50% and 75%.

However, such a method is found to be relatively aggressive with respect to the reactants and, in preference to this, a method by transesterification will be implemented, which method can be carried out under milder conditions, with the backbone being particularly well respected, thereby making it possible to obtain products which are of higher purity, and therefore not colored.

As indicated above, for the preparation of the products of the invention, use will preferably be made of a transesterification process.

This transesterification will advantageously be carried out using esters of C₈ to C₂₈ fatty acids and of C₁ to C₆ alcohols or mixtures of such esters, and will make it possible to graft the fatty chains originating from said esters by means of an ester type bond onto at least a part of the hydroxyl groups of the ulvan.

By way of examples of fatty esters that can be used to carry out such a transesterification, mention will be made of methyl, ethyl, isopropyl, pentyl, methyl, propyl, butyl or hexyl esters of fatty acids such as lauric acid, oleic acid, capric acid, myristic acid, palmitic acid, palmitoleic acid, linoleic acid, linolenic acid or arachidic acid, and also the mixtures of these various esters.

These esters may have various sources, in particular plant or animal sources. Mention will in particular be made of the following plant oils: soybean oil, groundnut oil, sunflower oil, rapeseed oil, sesame oil, olive oil, palm oil, cabbage palm oil, coconut oil, linseed oil and castor oil, fish animal oils, butters, lard and tallow.

According to a first variant, the transesterification may be carried out in a solvent medium.

By way of nonlimiting examples of solvents used in such a method of transesterification, mention will be made of dimethyl sulfoxide, diethyl sulfoxide, dibutyl sulfoxide, dibenzyl sulfoxide, N,N-dimethylformamide, N,N-diethylformamide and N,N-diphenylformamide.

The solvent used to carry out the transesterification will subsequently have to be eliminated, for example by evaporation.

The elimination of the solvent will have to be more or less complete, depending on the use subsequently intended for the product of the invention.

According to another preferred variant of the invention, the product may be prepared by transesterification without solvent.

Such a method proves to be much more advantageous than the previous method for industrial purposes since it is less expensive and avoids the problems linked to the use of the solvent and to the need, in certain cases, to eliminate all the solvent.

In general, the methods of transesterification in a nonsolvent medium, developed in the context of the present invention, are based on the methods described in U.S. Pat. No. 4,517,360 and U.S. Pat. No. 963,699, but have required a certain number of adaptations linked in particular to the nature of the specific substrate used for the grafting of the ester functions in the case of the products of the invention.

For the purpose of the invention, the expression “transesterification in a nonsolvent medium” is intended to mean that the transesterification reaction itself is carried out in the absence of solvent. However, the recovery of the product formed during the reaction will require the use of at least one solvent medium in one or more extraction steps, as emerges from the detailed description which follows.

The method of transesterification without solvent is advantageously carried out by bringing the polysaccharide into contact with at least one fatty ester as defined above and at least one soap-based compound, advantageously at least one compound based on alkali metal soaps of saturated or unsaturated fatty acids.

The presence of this or these soap(s) makes it possible to greatly improve the intersolubility between the two ulvan and fatty ester reactants.

The term “alkali metal soaps of fatty acids” is intended to mean the alkali metal soaps of fatty acids containing from 8 to 22 carbon atoms. Examples of alkali metal soaps of fatty acids are in particular the sodium, lithium, potassium, rubidium and cesium salts of capric acid, lauric acid, oleic acid, myristic acid, palmitic acid, licanic acid, parinaric acid, arachidic acid or stearic acid, and also mixtures thereof.

The reactants described above for the preparation of the transesterification product form a heterogeneous mixture.

The precise ratios of reactants can be freely determined either by experimentation or by using the examples given hereinafter. In general, the starting mixture of reactants comprises from approximately 5% to approximately 75%, preferably from approximately 15% to approximately 35% by weight of ulva-derived polyoses, from approximately 20% to approximately 90%, preferably from approximately 40% to approximately 70% by weight of fatty acid esters and from approximately 1% to approximately 35%, preferably from approximately 10% to approximately 20% by weight of alkali metal soaps of fatty acids.

The heterogeneous mixture is heated to a temperature advantageously comprised between approximately 70° C. and approximately 180° C., preferably between approximately 100° C. and 150° C. under a pressure of approximately 0.1 mm of mercury to approximately 760 mm of mercury, preferably from 0.5 to 100 mm of mercury. With this temperature and pressure range, a homogeneous mixture of partially or totally esterified polyoses and of starting reactants that have not reacted is formed after a period ranging from approximately 1 hour to 18 hours, preferably a period ranging from approximately 4 hours to 10 hours.

Following this reaction step, an organic solvent, which may be either soluble or insoluble in water and the effect of which is to modify the viscosity of the reaction medium, will advantageously be added.

The addition of this solvent makes it possible to form a more fluid organic phase. The amount of solvents to be added depends greatly on the viscosity of the reaction medium at the end of the reaction step.

The reaction medium is subsequently acidified.

By way of example, mention may be made of several acids that can be used: hydrochloric acid, sulfuric acid, citric acid, lactic acid, acetic acid and formic acid, and mixtures thereof.

The objective of this treatment with an acid is to allow the elimination of the metal soap(s) by neutralization.

Next, a rapid and simple treatment makes it possible to provide a family of polyose-esters. This treatment involves adding water, allowing the formation of a gel.

The gel formed is separated from the reaction medium and then, if necessary, it can be dried by conventional drying techniques so as to give a family of polyose-esters.

The family of polyose-esters thus obtained is characterized by compounds with a fatty ester/polyose proportion which, by way of example, may be between 5% and 85% by weight, preferably between 25% and 70%. These products are readily water-soluble and give compounds which have advantageous properties, in particular hydrating, emulsifying and coemulsifying properties, and which give a soft and substantive feel in solution.

The method by transesterification in a nonsolvent medium as defined above makes it possible to recover at least a part of the polyose-esters formed in the transesterification reaction in the form of an aqueous gel.

This method has the advantage of being particularly simple.

However, depending on the length of the fatty chains attached to the backbone and/or on the degree of grafting, there are cases where, in order to recover as quantitatively as possible all the polyose-esters formed, it will be advantageous to follow the step of extraction in the form of a gel in an aqueous medium with a second step of extraction of the polyose-esters possibly contained in the organic phase previously formed.

This additional extraction step intended to give a better recovery of all the polyose-esters formed will prove to be particularly advantageous when the fatty chains are long or when the degree of grafting of these chains onto the polysaccharide backbone is high. This will in particular be the case when the acid chains contain 16 carbon atoms or more or when the degree of grafting is greater than or equal to 30%.

Such a treatment results in the separation of two families of polyose-esters, one of which has a higher average degree of grafting than the other.

In such a method, a method is first of all carried out which is in all respects identical to that described above in which the transesterification in a nonsolvent medium, preferably in the presence of at least one metal soap, is carried out, as defined above.

Then, as according to the above method, the metal soap is eliminated and the viscosity of the medium is adjusted before precipitating a first family of polyose-esters in the form of a gel.

Next, the gel formed is separated from the reaction medium and then, if necessary, it can be dried by conventional drying techniques and it thus gives a first family of polyose-esters.

The remaining reaction medium is washed one or more times with water and the washing water is extracted with a solvent of partially water-soluble or water-insoluble alcohol type, for example 1-butanol, 2-butanol, pentanol, hexanol or cyclohexanol. The alcoholic phase is subsequently dried and gives a second family of polyose-esters, which exhibits a degree of grafting greater than that of the first. The first family of polyose-esters is characterized by less-grafted compounds where, by way of example, the fatty ester/polyose proportion may be between 5% and 75% by weight, preferably between 20% and 60%.

This family is more hydrophilic and gives compounds which have advantageous properties, in particular emulsifying and coemulsifying properties, and which confer a soft and substantive feel in solution.

The second family is characterized by compounds having a relatively low degree of grafting, with a high fatty ester/polyose proportion, of between, by way of example, 15% and 100% by weight, preferably between 40% and 90%. This family has very marked hydrating properties.

The present invention also relates to the use of the polyose-fatty ester product as emulsifier or coemulsifier in the context of cosmetic or pharmaceutical topical applications.

In this use, any proportion of the above-mentioned product may be used.

Particularly preferred proportions by weight for which an emulsifying or coemulsifying effect is obtained range between 0.1% and 10%. Preferred proportions are of the order of 1% to 3% by weight relative to the total weight of the composition to be emulsified.

The invention also relates to the use of the polyose-fatty ester product as film-forming agent giving in particular a soft feel in the context of cosmetic or pharmaceutical applications.

In this use, any proportion of the above-mentioned complex product may be used.

Particularly preferred proportions by weight for which a film-forming and soft effect is obtained range between 0.1% and 25%. Preferred proportions are of the order of from 1% to 3% by weight relative to the total weight of the composition to be emulsified.

The invention also relates to the use of the product as moisturizer in the context of cosmetic or pharmaceutical applications. In this use, any proportion of the product of the invention may be used. Particularly preferred proportions by weight for which a moisturizing effect is obtained range between 0.1% and 10%. Preferred proportions are of the order of from 1% to 3% by weight relative to the total weight of the composition.

The invention also relates to an emulsifying composition, characterized in that it contains, as emulsifier or coemulsifier, a complex polyose-fatty ester product as defined above.

The invention also relates to a moisturizing composition, characterized in that it contains, as moisturizer, a polyose-fatty ester product as defined above.

The present invention also relates to a film-forming composition, characterized in that it contains, as film-forming agent, a polyose-fatty ester, as defined above.

Because of their notable surfactant properties, the products of the invention may also be used in a completely different field, namely the recovery or dispersion of hydrocarbons, both on soils and on expanses of water. In such applications, the product of the invention will be used as surfactant for emulsifying or dispersing fatty phases, at concentrations conventionally used for this purpose.

### EXAMPLES

In all the examples which follow, the extract of ulva treated is obtained according to the protocol described in Hydrobiologia 326/327: 473-480, 1996 (Hot extraction in an aqueous medium and ultrafiltration).

### Example 1

### Preparation of a Highly Esterified Polyose-Ester

300 g of ulva polysaccharides diluted in 5 liters of pyridine are mixed with 1 kg of lauryl chloride at a temperature of 130° C. for 2 hours. Mechanical stirring is fixed at 400 rpm. The reaction is subsequently stopped by adding a mixture of alcohol and water. The whole mixture is vacuum-filtered and washed with ethanol and with acetone. The compound obtained is highly grafted but also highly colored, which proves that there is a certain amount of degradation.

### Example 2

### Preparation of Two Families of Polyose-Esters from Methyl Oleate by Transesterification Without Solvent

The ulva-derived polyoses (500 grams), methyl oleate (1200 grams), sodium oleate (225 g) and lithium oleate (53 g) are mixed in a 4-liter reactor. The mixture is heated at 150° C. for 6 h with mechanical stirring (600 rpm) and under reduced pressure. The reaction medium is dissolved in butanone. The soaps are neutralized with lactic acid. Distilled water is added and a gelled phase is formed, which is readily separated from the reaction medium. This gelled phase is dried and constitutes the first family of polyose-esters. In parallel, the rest of the reaction medium is washed several times with distilled water and then the washing water is extracted with butanol. The butanolic phase is dried and contains the second family of polyose-esters. This second family contains the most highly grafted polyoses. Distillation of the remaining organic phase makes it possible to recover butanone, fatty acids and methyl esters for recycling.

### Example 3

### Preparation of Two Families of Polyose-Esters from Methyl Laurate by Transesterification Without Solvent

The ulva-derived polyoses (200 grams), methyl laurate (360 grams), sodium oleate (117 g) and lithium oleate (27.7 g) are mixed in a 2-liter reactor. The mixed is heated at 100° C. for 6 h with mechanical stirring (600 rpm) and under reduced pressure. The reaction medium is dissolved in butanone. The soaps are neutralized with lactic acid. Distilled water is added and a gelled phase is formed, which is readily separated from the reaction medium. This gelled phase is dried and constitutes the first family of polyose-esters. In parallel, the rest of the reaction medium is washed several times with distilled water and then the washing water is extracted with butanol. The butanolic phase is dried and contains the second family of polyose-esters. This second family contains the most highly grafted polyoses. Distillation of the remaining organic phase makes it possible to recover butanone, fatty acids and methyl esters for recycling.

### Example 4

### Preparation of a Family of Polyose-Esters from Methyl Oleate by Transesterification without Solvent

The ulva-derived polyoses (500 grams), methyl oleate (1200 grams), sodium oleate (225 g) and lithium oleate (53 g) are mixed in a 4-liter reactor. The mixture is heated at 150° C. for 6 h with mechanical stirring (600 rpm) and under reduced pressure. The reaction medium is dissolved in butanone. The soaps are neutralized with lactic acid. Distilled water is added and the aqueous phase containing a gel is separated. This phase is dried and constitutes the complex family of polyose-esters. Distillation of the remaining organic phase makes it possible to recover butanone, fatty acids and methyl esters for recycling. The polyose-esters have a fatty acid/polyose proportion which is close to 55% by weight.

### Example 5

### Preparation of a Family of Polyose-Esters from Methyl Laurate by Transesterification without Solvent

The ulva-derived polyoses (200 grams), methyl laurate (360 grams), sodium oleate (117 g) and lithium oleate (27.7 g) are mixed in a 2-liter reactor. The mixture is heated at 100° C. for 6 h with mechanical stirring (600 rpm) and under reduced pressure. The reaction medium is dissolved in butanone. The soaps are neutralized with lactic acid. Distilled water is added and the aqueous phase containing a gel is separated. This phase is dried and constitutes the complex family of polyose-esters. Distillation of the remaining organic phase makes it possible to recover butanone, fatty acids and methyl esters for recycling. The polyose-esters have a fatty acid/polyose proportion which is close to 40% by weight.

### Example 6

### Simple Formulation for Applications as Shampoo

Texapon NSO (sodium laureth sulfate) 20.0% by weight

Tegobetaine F 50 (cocamidopropylbetaine) 5% by weight

Polyose-ester complex derived from example 4: 1.5% by weight

NaCl quantity sufficient for the desired viscosity

Water: to make up the remainder to 100.0% by weight.

The shampoo is homogeneous, stable and soft to the touch after rinsing.

A shampoo which is similar in all respects but which does not contain the complex of example 4 gives a dry feel after rinsing.

### Example 7

### Simple Formulation for Applications as Shower Gel

Texapon NSO (sodium laureth sulfate) 20.0%

Tegobetaine F 50 (cocamidopropylbetaine) 5%

Polyose-ester complex of example 5: 2.5%

NaCl quantity sufficient for viscosity

Water: to make up the remainder to 100.0%

The shower gel is homogeneous and stable and leaves a soft and pleasant film on the skin after rinsing.

A shampoo which is similar in all respects but which does not contain the complex of example 4 gives a dry feel after rinsing

### Example 8

### Emulsion Formulation

Polyose-ester compound of example 4: 2.5%

Polysorbate 80: 4%

Capric/caprylic triglyceride: 6%

Stearic acid: 10%

Water: to make up the remainder to 100%

An excellent homogeneous and stable emulsion is obtained. The polyose-ester compound of example 4 is therefore an emulsifying compound. In addition, it gives a soft and substantive feel.

### Example 9

### Emulsion Formulation

Polyose-ester compound of example 5: 2.5%

Capric/caprylic triglyceride: 10%

Span 60 (sorbitan stearate): 3%

Water: to make up the remainder to 100%

An excellent, homogeneous and stable emulsion is obtained. The polyose-ester compound of example 5 is therefore a coemulsifying compound. In addition, it gives a soft and substantive feel.

1. A product resulting from the grafting, by esterification or transesterification, onto at least a part of the hydroxyl functions of an ulvan-type polysaccharide in the form of an acid or in the form of a mono- or divalent salt of fatty chains or of mixtures of fatty chains containing 8 to 28 carbon atoms, said fatty chains being saturated or unsaturated, and linear or branched.

2. The product as claimed in claim 1, wherein said ulvan-type polysaccharide is in the form of a mono- or divalent salt.

3. A method for synthesizing products or mixtures containing at least one product as defined in claim 1, wherein it comprises a step of esterification or transesterification of at least a part of the hydroxyl functions of an ulvan-type polysaccharide or of a salt thereof, obtained by extraction from an alga of ulva or enteromorph type or from a mixture of these algae.

4. The method as claimed in claim 3, wherein it is carried out on an extract of ulva or enteromorph in solid form containing at least 85%, preferably at least 95% by weight of ulvan.

5. The method as claimed in claim 3, wherein it comprises a step of esterification by means of an acid chloride of an acid comprising a fatty chain containing from 8 to 28 carbon atoms.

6. The method as claimed in claim 3, wherein it comprises a step of grafting of fatty chains as defined in claim 1, by transesterification, onto at least a part of the hydroxyl groups of said ulvan, using esters of a C₈-C₂₈ fatty acid and of a C₁-C₆ alcohol, or mixtures of such esters.

7. The method as claimed in claim 6, wherein said transesterification is carried out in a solvent medium, said solvent subsequently being eliminated by evaporation.

8. The method as claimed in claim 6, wherein said transesterification is carried out in a nonsolvent medium.

9. The method as claimed in claim 8, wherein a solvent intended to adjust the viscosity of the reaction medium is added.

10. The method as claimed in claim 8, wherein said transesterification is carried out in the presence of a metal soap or a mixture of metal soaps.

11. The method as claimed in claim 10, wherein said metal soap is subsequently eliminated by neutralization with an acid.

12. The method as claimed in claim 8, wherein it comprises a step of extraction by means of at least one solvent.

13. The method as claimed in claim 12, wherein it comprises at least one step of extraction with an aqueous medium in order to recover at least a part of the product resulting from said transesterification in the form of an aqueous gel.

14. The method as claimed in claim 13, wherein it also comprises an extraction step carried out by means of an organic solvent for the product not extracted during the extraction with said aqueous medium.

15. (canceled)

16. The method as claimed in claim 19, wherein said product is used as emulsifier.

17. The method as claimed in claim 19, wherein said product is used in a cosmetic or pharmaceutical composition for topical application, as agent which confers a soft and/or substantive feel and/or as moisturizer and/or as agent which confers film-forming properties.

18. The method as claimed in claim 19, wherein said product is used in a composition for recovering or dispersing hydrocarbons on the ground or on an expanse of water.

19. A method of providing a surfactant effect comprising:
applying a material comprising the product of claim 1 in an environment in which a surfactant effect is desired.
